(19) United States
(12) Patent Application Publication
(10) Pub. No.: US 2006/0269441 A1
(43) Pub. Date: Nov. 30, 2006

(54) Nanosilica-based food contact sanitizer

(76) Inventors: Maria G. Ochomogo, Pleasanton, CA (US); Michael J. Petrin, Pleasanton, CA (US); Lafayette D. Foland, Pleasanton, CA (US)

Correspondence Address: THE CLOROX COMPANY, P.O. BOX 24305, OAKLAND, CA, 94623-1305, US

(21) Appl. No.: 11/139,316

(22) Filed: May 25, 2005

(51) Int. Cl. A61L 2/18

(52) U.S. Cl. 422/28

(57) ABSTRACT

Described herein are nanoparticle silica Pickering emulsions comprising an aqueous suspension of colloidal silica mixed with a quaternary ammonium biocide. Additionally, the composition may include a small amount of organic solvent; such as methanol, ethanol, isopropanol, and mixtures thereof; wetting agents, such as a silicone-based wetting agent; and organic bases which act as solvent buffers, if it is desirable to raise the pH of the composition, such as monoethanolamine (MEA). The combination of nanoparticle silica and quaternary ammonium biocide provides food contact sanitization at less than 200 ppm of quaternary ammonium active.

### BACKGROUND OF THE INVENTION

1. Field of the Invention

The invention relates to the use of nanoparticles with amphipathic properties to synergistically interact with functional biocidal materials in aqueous solutions. The invention is directed generally to a low active level biocidal hard surface food contact sanitizer composition and a method of forming the same.

This invention relates to the special formulation of sanitizer as approved by the Food Industry and United States Food and Drug Administration (FDA) to provide for the proper cleaning and sanitizing of food-related contact surfaces, utensils and containers when used with the preparation of food in accordance with the “sanitizer solution standards” as established by U.S. Federal Government, the FDA as well as H.A.C.C.P. (Hazard Analysis Critical Control Point) regulations which includes three sanitizers approved for food industry use being quaternary ammonium concentration solution, chlorine/bleach concentration solution and iodine concentration solution all of which have been tested, qualified and approved by the Environmental Protection Agency of the United States (EPA); U.S. Food and Drug Administration (FDA) under 21 CFR 178.1010; the United States Department of Agriculture (USDA) and the United States Department of Commerce (USDC). Accordingly, these four agencies are involved for the general public's safety and protection from pathogens and the protection of food from spoilage.

2. Description of the Related Art

Current commercially available biocidal disinfectants generally employ a germicidal active present at a concentration sufficient to achieve complete kill of targeted microorganisms on a surface within a specified time period, often in the presence of a soil load. When quaternary ammonium disinfectants (“quats”) are employed, the levels required typically exceed 200 ppm (parts per million), a level that corresponds to that Generally Recognized As Safe (G.R.A.S.) for use on food contact and food preparation areas by the United States Environmental Protection Agency (U.S.E.P.A.).

When quaternary ammonium disinfectants are employed at higher levels, a separate rinsing step following application is generally recommended to remove excess quat before the surface can be used or contacted. In addition, even when a rinsing step is not needed, for example in the disinfection of other surfaces, and particularly glossy surfaces such as glass, tiles and metal, higher levels of quat tend to leave visibly apparent films on the surfaces. When higher levels of quat are employed in order to achieve extended disinfectancy, the deposited levels may further exhibit tackiness leading to unpleasant tactile characteristics on the treated surfaces.

Accordingly, there is a need for an effective hard surface disinfectant that contains biocidal actives at levels below 200 ppm.

### SUMMARY OF THE INVENTION

One aspect of the invention provides a method of preparing a hard surface food contact sanitizer composition comprising: (a) providing an aqueous colloidal dispersion of nanoparticle-sized silica, wherein the silica particles have a particle size distribution range of 1-100 nm; (b) providing at least one quaternary ammonium biocide, wherein the level of quaternary ammonium biocide present in the food contact sanitizer composition necessary to accomplish sanitization according to EPA standards is less than the level of quaternary ammonium biocide that would be necessary absent the colloidal dispersion of nanoparticle-sized silica; and (c) combining the colloidal dispersion with the at least one quaternary ammonium biocide such that the concentration of silica ranges from 0.05 to 10 ppm and the concentration of biocide ranges from 10 to 1000 ppm.

A further aspect of the invention provides a method of sanitizing a hard surface to come in contact with food comprising the steps of: (1) contacting a hard surface with a spray containing or a non-woven wipe impregnated with a food contact sanitizer composition comprising: (a) an aqueous colloidal dispersion of nanoparticle-sized silica, wherein the average particle size of the silica particles ranges from 1 to 100 nm, and (b) at least one quaternary ammonium biocide, wherein the level of quaternary ammonium biocide present in said food contact sanitizer composition necessary to accomplish sanitization according to EPA standards is less than the level of quaternary ammonium biocide that would be necessary absent said aqueous colloidal dispersion of nanoparticle-sized silica; and (2) wiping said sprayed surface or wiping said surface with the non-woven wipe such that a thin film residue of said food contact sanitizer composition remains on the hard surface, wherein said quaternary ammonium biocide residue without rinsing remaining on the hard surface is less than 200 ppm.

Another aspect of the invention includes a hard surface food contact sanitizer composition comprising: an aqueous colloidal dispersion of nanoparticle-sized silica, wherein the average particle size of the silica particles ranges from 1 to 100 nm; and at least one quaternary ammonium biocide, wherein the level of quaternary ammonium biocide present in the food contact sanitizer composition necessary to accomplish sanitization according to EPA standards is less than the level of quaternary ammonium biocide that would be necessary absent the colloidal dispersion of nanoparticle-sized silica.

Further features and advantages of the present invention will become apparent to those of ordinary skill in the art in view of the detailed description of preferred embodiments below, when considered together with the attached drawings and claims.

### Definitions

In this document, use shall be made of the following terms of art, which have the meanings indicated below.

As used herein the terms “biocide” and “biocidal” shall refer to any substance that is capable of destroying living organisms including, for example, germicides, disinfectants, antivirals, sanitizers, pesticides, microbiocidals, sterilant, antibiotics, bactericides, fungicides, and/or any substance that is capable of preventing the growth of archaea, bacteria, yeast, fungus, virus or any combination thereof.

As used herein the term no rinse food contact sanitizer is defined as a sanitizing composition that kills Escherichia coli ATCC #11229 (E. Coli) and Staphylococcus aureus ATCC #6538 (Staph A.) after a 30 second contact time leaving a residue of less than 200 ppm biocide with no rinsing required.

The term “Pickering emulsion” refers to a particle-stabilized emulsion. “Modified Pickering emulsion” refers to a Pickering emulsion that picks up soils and other substances as it is used and thus, has been “modified” by incorporating those soils and other substances into the emulsion.

“Amphipathic properties” as used herein means particles capable of behaving in both a hydrophilic and a hydrophobic manner.

### BRIEF DESCRIPTION OF THE DRAWINGS

FIG. 1 shows the structure of a generic quaternary ammonium biocide.

FIG. 2 illustrates a possible mechanism that results in improved quat efficacy in the presence of a nanoparticle silica suspension.

FIG. 3 is a bar graph showing the results of a Food Contact Sanitization test.

FIG. 4 is a phase diagram showing a mixture contour plot of Bindzil and Barquat 4250Z.

FIG. 5 is a phase diagram showing a mixture contour plot of Klebosol and Barquat 4250Z.

### DETAILED DESCRIPTION

Before describing embodiments in detail, it is to be understood that the terminology used herein is for the purpose of describing particular embodiments, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

All numbers expressing quantities of ingredients, constituents, reaction conditions, and so forth used in the specification and claims are to be understood as being modified in all instances by the term “about”. Notwithstanding that the numerical ranges and parameters setting forth the broad scope of the subject matter presented herein are approximations, the numerical values set forth in the specific examples are reported as precisely as possible. Any numerical value, however, inherently contains certain errors necessarily resulting from the standard deviation found in the respective testing measurements.

The invention relates to the use of nanoparticles with amphipathic properties that synergistically interact with functional biocidal materials in aqueous solutions allowing the effective amount of functional material to be significantly reduced. It is expected that other combinations of actives with amphipathic nano-particles capable of forming Pickering emulsions will show similar benefits with respect to suspension, stabilization and delivery of materials from homogeneous, stable, aqueous solution to target surfaces and materials. Suitable materials are anticipated to include other biocides, perfumes, active oils, low or non-water soluble ingredients, surfactants, and the like. Specifically, embodiments herein are illustrated in the context of a hard surface food contact sanitizer composition with a particular emphasis on food contact sanitization. The skilled artisan will readily appreciate, however, that the materials and methods disclosed herein will have application in a number of other contexts where biocidal activity is desirable, particularly where low levels of the biocidally active component is important.

A nanoparticle silica Pickering emulsion comprising an aqueous suspension of colloidal silica mixed with a quaternary ammonium biocide is described herein. Additionally, the composition may include a small amounts of water-miscible organic solvents; such as the mono-protic alcohols including methanol, ethanol, isopropanol, and the multi-protic glycols and/or glycol ethers; wetting agents, such as a silicone-based wetting agent; and organic bases which act as solvent buffers, if it is desirable to raise the pH of the composition, such as monoethanolamine (MEA), diethanolamine (DEA), triethanolamine (TEA), ammonia and ammonium derivatives. As will be discussed, the combination of nanoparticle silica and quaternary ammonium biocide, hereinafter referred to as “quat”, provides food contact sanitization at less than 200 ppm of quat active. Additionally, all the components of the sanitizing composition embodiments described herein are GRAS (Generally Regard As Safe).

Without being bound by any particular theory, it is believed that the use of nano-particles with amphipathic properties and with sizes in the range of between about 1-100 nanometers create a synergistic interaction with certain active ingredients, e.g., quats, that enhance the active ingredient's functionality. It is believed that the amphipathic surface properties enable the nano-particles to act as carriers of the actives reversibly and/or releasably bound to their surfaces. The ratio of silica to quat can range from 5×10⁻⁵:1 to 1:1. One example is a nano-suspension of colloidal silica combined with a quat (e.g., benzyl dialkyl quaternary ammonium chloride, Barquat 4250Z). It is possible that the nanoparticles of silica are modified by the quat forming a silica/quat web for cleaning and enhanced killing of gram negative bacteria such as E. Coli. This combination of colloidal silica and quat provides food contact sanitization on hard surfaces at 100 ppm levels, which is well below the 200 ppm quat maximum currently approved for food contact sanitization on hard surfaces by the EPA. The colloidal silica surprisingly enhances the germicidal effect of the quat as well as its cleaning efficiency.

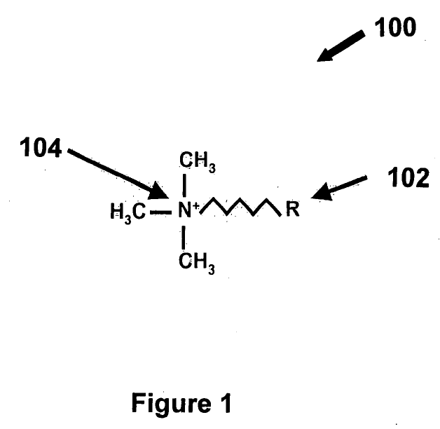

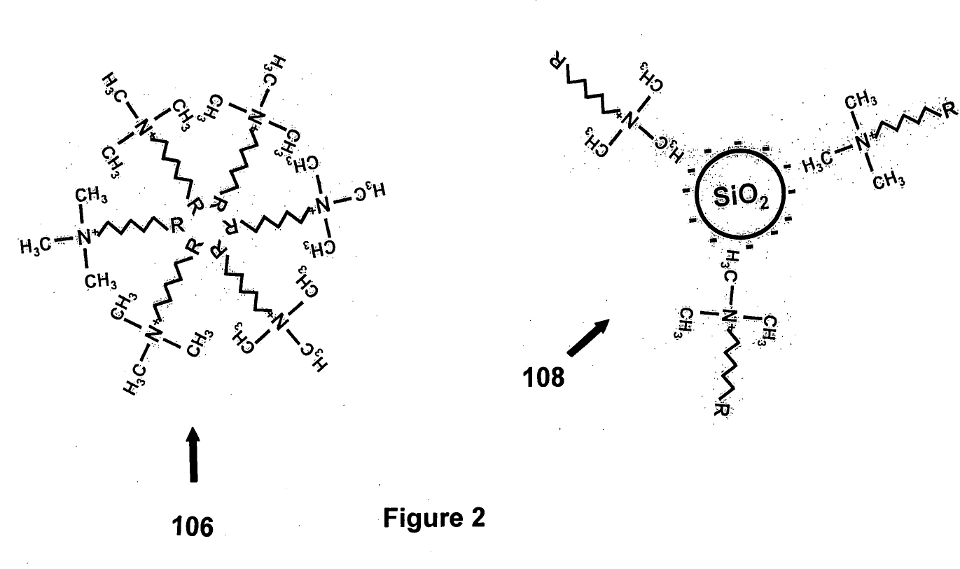

The improvement observed is believed to be the result of the particle size of the silica particles (1-100 nm) combined with their amphipathic property. Interaction with the cationically charged quat is favored thus forming a modified Pickering emulsion. Referring to FIG. 1, a general structure for a quaternary ammonium biocide molecule 100 is shown. The molecule has a hydrophobic biocide portion 102 and a hydrophilic quaternary ammonium portion 104. Referring to FIG. 2, it is believed that the quats tend to form hydrophilic clusters 106 when not in the presence of amphipathic nanoparticles which limit the amount of biocide cites available to react with bacteria present on a surface. In contrast, quats in the presence of amphipathic nanoparticles tend to adhere to the nanoparticles forming quaternary ammonium-modified nanoparticles 108, hereinafter referred to as quat-modified nanoparticles. These quat-modified nanoparticles cause the biocidal portion of the quat to be distributed by the nanoparticles thereby reducing the amount of quat-clusters formed which results in more biocidally-effective sites to be available. The quat-modified nano-particles are believed to be penetrating the bacteria's liposaccharide cell membrane resulting in enhanced biocidal potency of the combined system versus use of either material alone. There is evidence that colloidal silica immobilizes the cytoplasm (W. Worthy, Chem. Eng. News, 17 Feb. 9, 1977), and in the present discovery, it is believed that the quat-modified nano-particles are enabled to more effectively deliver the quat into the cytoplasm and/or act directly to disrupt the membrane resulting in synergistically enhanced biocidal efficacy. Abrasion from particle sizes that are about 50 nm or larger could also influence cleaning and provide frictional or polishing action as well on the hard surfaces to provide the improved soil and residue removal observed.

It is believed that other combinations of actives with amphipathic nano-particles capable of forming Pickering emulsions will show similar benefits with respect to suspension, stabilization and delivery of materials from homogeneous, stable, aqueous solutions to target surfaces and materials. Suitable materials are anticipated to include other biocides, perfumes, active oils, low or non-water soluble ingredients, surfactants, and the like.

Materials

The sanitizing composition of the present invention can be dispensed in a variety of methods, both independently from or in conjunction with an absorbent material. The sanitizing composition can be specifically formulated to be loaded onto a wipe substrate which wipe substrate includes wood pulp and/or wood pulp derivatives and will be described with particular reference thereto.

As discussed, the wipe substrate is generally an absorbent material. Preferably, it is a nonwoven sheet, which is at least one layer, made of wood pulp; or a blend of wood pulp and a synthetic fiber, without limitation, such as polyester, rayon, nylon, polypropylene, polyethylene, other cellulose polymers; or a synthetic fiber or mixture of such fibers. The nonwovens may include nonwoven fibrous sheet materials which include meltblown, coform, air-laid, spun bond, wet laid, bonded-carded web materials, hydroentangled (also known as spunlaced) materials, and combinations thereof. These materials can comprise synthetic or natural fibers or combinations thereof. A binder may or may not be present. Manufacturers include Kimberly-Clark, E.I. du Pont de Nemours and Company, Dexter, American Nonwovens, James River, BBA Nonwovens and PGI. Examples of such wipe substrates are depicted in: Bouchette et al., U.S. Pat. Nos. 4,781,974 and 4,615,937, Clark et al., U.S. Pat. No. 4,666,621, Amundson et al., WO 98/03713, and Cabell et al., U.S. Pat. No. 5,908,707, Mackey et al., WO 97/40814, Mackey et al., WO 96/14835 and Moore, EP 750063, all of which are incorporated herein by reference.

Woven materials, such as cotton fibers, cotton/nylon blends, or other textiles may also be used herein. Regenerated cellulose, polyurethane foams, and the like, which are used in making sponges, may also be suitable for use herein.

The wipe substrate's liquid loading capacity should be at least about 50%-1000% of the dry weight thereof, typically in the range of at least about 200%-800%. This is expressed as loading ½ to 10 times the weight (or, more accurately, the mass) of the wipe substrate. The wipe substrate varies without limitation from about 0.01 to about 1,000 grams per square meter (g/m²), generally from 25 to 120 g/m² (referred to as “basis weight”) and typically is produced as a sheet or web which is cut, die-cut, or otherwise sized into the appropriate shape and size. The wipe substrates, which are now referred to simply as wipes, can be individually sealed with a heat-sealable or glueable thermoplastic overwrap (such as polyethylene, Mylar, and the like). In one embodiment the wipes can be packaged as numerous, individual sheets which are then impregnated or contacted with the liquid sanitizing ingredients of the invention for more economical dispensing. In another embodiment, the wipes can be formed as a continuous web during the manufacturing process and loaded into a dispenser, such as a canister with a closure, or a tub with closure. The closure is to seal the moist wipes from the external environment and to prevent premature volatilization of the liquid ingredients. Without limitation, the dispenser may be formed of plastic, such as high density polyethylene, polypropylene, polycarbonate, polyethylene phtherethalate (PET), polyvinyl chloride (PVC), or other rigid plastics. The continuous web of wipes could preferably be threaded through a thin opening in the top of the dispenser, most preferably, through the closure. A means of sizing the desired length or size of the wipe from the web would then be needed. A knife blade, serrated edge, or other means of cutting the web to desired size can be provided on the top of the dispenser, for non-limiting example, with the thin opening actually doubling in duty as a cutting edge. Alternatively, the continuous web of wipes could be scored, folded, segmented, or partially cut into uniform or non-uniform sizes or lengths, which would then obviate the need for a sharp cutting edge. Further, as in hand tissues, the wipes could be interleaved, so that the removal of one wipe advances the next, and so forth.

The wipes will have a certain wet tensile strength which is without limitation about 25 to about 250 Newtons/m, more preferably about 75-170 Newtons/m.

The sanitizing composition can be loaded onto a wipe which is made of an absorbent/adsorbent material. Typically, the wipe has at least one layer of nonwoven material. Nonlimiting examples of commercially available cleaning wipes that can be used include DuPont 8838, Dexter ZA, Dexter 10180, Dexter M10201. All of these wipes include a blend of polyester and wood pulp. Dexter M10201 also includes rayon, a wood pulp derivative. The loading ratio of the sanitizing composition onto the wipe is about 2-5 to 1, and typically about 3-4 to 1. The sanitizing composition is loaded onto the wipe in any number of manufacturing methods. Typically, the wipe is soaked in the cleaning composition for a period of time until the desired amount of loading is achieved. The wipe loaded with the sanitizing composition provides excellent sanitizing with little or no streaking/filming. The wipe(s) can be packaged individually or in groups.

The Sanitizing Composition:

The sanitizing composition can contain between 0.05-10 ppm of silica and 10-1000 ppm of biocide. The sanitizing composition is impregnated, dosed, loaded, metered, or otherwise dispensed onto the wipe. This can be executed in numerous ways. For example, each individual wipe could be treated with a discrete amount of sanitizing composition. More preferably, a mass treatment of a continuous web of wipes with the sanitizing composition will ensue. In some cases, an entire web of wipes could be soaked in the cleaner. In other cases, while the web is being spooled, or even during the creation of the nonwoven material, the sanitizing composition could be sprayed or otherwise metered onto the web. A mass, such as a stack of individually cut and sized wipes could also be impregnated in its container by the manufacturer, or, even by the user.

The Nano-Particle Silica Dispersion:

Nanoparticles, defined as particles with diameters of about 400 nm or less, are technologically significant, since they have novel and useful properties due to the very small dimensions of their particulate constituents. “Non-photoactive” nanoparticles do not use UV or visible light to produce the desired effects. Nanoparticles can have many different particle shapes. Shapes of nanoparticles can include, but are not limited to spherical, parallelepiped-shaped, tube shaped, and disc or plate shaped.

Nanoparticles with particle sizes ranging from about 1 nm to about 400 nm can be economically produced. Particle size distributions of the nanoparticles may fall anywhere within the range from about 1 nm, or less, to less than about 400 nm, alternatively from about 2 nm to less than about 300 nm, and alternatively from about 5 nm to less than about 150 nm. Commercial colloidal silica suspensions having a primary particle size between 5 to 150 nanometer (nm) and a surface area between 50-800 m²/g are suitable for use in the present invention. The surface area is generally measured by BET (see DIN 66131; originally described in JACS, Vol. 60, 1938, p. 309 by Brunauer, et al. Colloidal suspensions are generally preferred for ease of handling in preparing the inventive compositions, but these may also be prepared using any available source of colloidal silica according to methods known in the art.

The source of colloidal silica may be selected from silica dioxide, silicon dioxide, crystalline silica, quartz, amorphous fumed silica, food grade silica, flint, hydrophobic fumed silica, treated fumed silica, untreated fumed silica, amorphous fused silica, precipitated amorphous silica, microcrystalline silica, foundry sand, utility sand, fracturing sand, silica sand, silica, flint, glass sand, melting sand, engine sand, blasting sand, traction sand, hydraulic fracturing sands, filter sand, soft silica, condensed silica fume, cristobalite, tridymite, synthetic fused silica, hydrated precipitated silica, colloidal silica, silica dispersion, and silica aerogels. Further, silicas may be selected from the general categories of silicone dioxide (SiO₂) described as aerogel, amorphous, colloidal, crystalline, diatomaceous, food grade, fumed, fused, hydrophilic, hydrophobic, novaculite, precipitated, quartz and/or synthetic silica. Amorphous (CAS #7631-86-9), crystalline (CAS # 14808-60-7), and/or mixed type colloidal silica particles may be employed. Generally, amorphous silica forms are preferably employed for applications in which their improved safety characteristics are desirable. Also suitable is amorphous fumed silica, crystalline-free (CAS # 112945-52-5), amorphous hydrated silica and synthetic amorphous silica gel (SiO₂xH2O, x=degree of hydration, CAS # 63231-67-4), precipitated silica gel, crystalline-free (CAS # 112926-00-8), amorphous, precipitated silica gel (CAS #7699-41-4), silica hydrate (CAS #10279-57-9), vitreous silica (CAS # 60676-86-0) and crystalline-free silicon dioxide (CAS #7631-86-9).

Suitable amorphous silicas commercially available in the preferred colloidal nanometer size domain include Ludox (available from Dupont), Klebosol (available from Clariant), Bindzil, Nyacol (both available from Akzo Nobel), Levasil (available from Bayer), Koestrosol (available from CWK), and Snowtex (available from Nissan Chemicals). For example, two varying sized colloidal silica products were evaluated, Bindzil 30/360FG (12 nm), 0.075 ppm and Klebosol 35 V 50 (70 nm), 0.10 ppm.

The Biocide:

The biocide in the sanitizing composition includes a cationic compound, typically comprising one or more quaternary ammonium compounds (quats). Quats are desirable in that such compounds have a broad spectrum of antimicrobial or biocidal properties. A variety of different quats can be used in the sanitizing composition. Suitable for use as the biocidal constituent is at least one cationic surfactant which is found to provide a broad antibacterial or sanitizing function. Any cationic surfactant which satisfies these requirements may be used and is considered to be within the scope of the present invention. Mixtures of two or more cationic surface active agents may also be used. Cationic surfactants are well known, and useful cationic surfactants may be one or more of those described for example in McCutcheon's Detergents and Emulsifiers, North American Edition, 1982; Kirk-Othmer, Encyclopedia of Chemical Technology, 3rd Ed., Vol. 22, pp. 346-387, the contents of which are herein incorporated by reference.

Examples of suitable cationic surfactant compositions useful in the practice of the instant invention are those which provide a biocidal effect to the concentrated compositions, including those quaternary ammonium compounds and salts thereof, which may be characterized by the general structural formula:
(R1R2R3R4)N+X−
wherein each of R1, R2, R3 and R4 is independently selected from an alkyl, aryl, alkylaryl or alkoxylated substituent of from 1 to 26 carbon atoms, and the entire cation portion of the molecule has a molecular weight of at least 165. The alkyl substituents may be long-chain alkyl, long-chain alkoxyaryl, long-chain alkylaryl, halogen-substituted long-chain alkylaryl, long-chain alkylphenoxyalkyl, arylalkyl, etc. The remaining substituents on the nitrogen atoms other than the abovementioned alkyl substituents are hydrocarbons, usually containing no more than 12 carbon atoms. The substituents R1, R2, R3 and R4 may be straight-chained or may be branched, but are preferably straight-chained, and may include one or more amide, ether or ester linkages. The counterion X may be any salt-forming anion which permits water solubility of the quaternary ammonium complex.

Exemplary quaternary ammonium salts within the above description include the alkyl ammonium halides such as cetyl trimethyl ammonium bromide, alkyl aryl ammonium halides such as octadecyl dimethyl benzyl ammonium bromide, N-alkyl pyridinium halides such as N-cetyl pyridinium bromide, and the like. Other suitable types of quaternary ammonium salts include those in which the molecule contains either amide, ether or ester linkages such as octyl phenoxy ethoxy ethyl dimethyl benzyl ammonium chloride, N-(laurylcocoaminoformylmethyl)-pyridinium chloride, and the like. Other very effective types of quaternary ammonium compounds which are useful as biocides include those in which the hydrophobic radical is characterized by a substituted aromatic nucleus as in the case of lauryloxyphenyltrimethyl ammonium chloride, cetylaminophenyltrimethyl ammonium methosulfate, dodecylphenyltrimethyl ammonium methosulfate, dodecylbenzyltrimethyl ammonium chloride, chlorinated dodecylbenzyltrimethyl ammonium chloride, and the like.

Quaternary ammonium compounds which act as biocides and which are be found useful in the practice of the present invention include those in which R1 is selected as herein above, R2, R3 and R4 are the same or different C8-C12 alkyl; or R2 is C12-16 alkyl, C8-18 alkylethoxy, C8-18 alkylphenolethoxy and R3 is benzyl; and X is a halide, for example chloride, bromide or iodide, or is a methosulfate anion. The alkyl groups recited in R2 and R3 may be straight-chained or branched, but are generally substantially linear.

Further examples include alkoxylated quaternary ammonium compounds wherein any one or more of R1, R2, R3 and R4 is a C8-C24 alkyl group containing an alkoxylated moiety.

Exemplary materials include ETHOQUAD 18/12 described to be octadecylmethyl [ethoxylated (2)]-ammonium chloride; ETHOQUAD 18/25, described to be octadecyl methyl [ethoxylated (15)] ammonium chloride, ETHOQUAD C/25, described to be coco methyl [ethoxylated (15)] ammonium chloride, ETHOQUAD C/12, described to be coco methyl [ethoxylated (2)] ammonium chloride; ETHOQUAD C/12 Nitrate, described to be coco methyl [ethoxylated (2)] ammonium nitrate; ETHOQUAD O/25, described to be oleyl methyl [ethoxylated (15)] ammonium chloride; ETHOQUAD O/12 described to be oleyl methyl [ethoxylated (2)] ammonium chloride; as well as ETHOQUAD T/12 described to be tallow alkyl methyl [ethoxylated (2)] ammonium chloride.

Further exemplary materials include Q-18-15 described to be octadecyl poly(15)oxyethylene methyl ammonium chloride, and Q-C-15, described to be coco poly(15)oxyethylene methyl ammonium chloride (both of which are available from Tomah Inc.); as well as VARIQUAT K-1215, a methyl bis-(polyethoxy ethanol) coco ammonium chloride, with an 15 ethoxy groups; ADOGEN 66, an ethyl bis-(polyethoxy ethanol) tallow ammonium chloride, having 15 ethoxy groups; VARISOFT 5TD, an ethoxylated di (C12-C18) alkyl methyl ammonium chloride, with 5 ethoxy groups; REWOQUAT CPEM, a coco pentaethoxy methyl ammonium methosulfate, with 5 ethoxy groups.

Particularly useful quaternary biocides include compositions which include a single quaternary compound, as well as mixtures of two or more different quaternary compounds. Particularly useful quaternary biocides include those which are described as being a blend of alkyl dimethyl benzyl ammonium chlorides; BARDAC® 205M, BARDAC® 2050, BARDAC® 2080, BARDAC® 2250, BTC® 812, BTC® 818 and BTC® 1010, which are described as being based on dialkyl(C8-C10)dimethyl ammonium chloride; BARDAC® 2250 and BARDAC® 2280 or BTC® 1010, which are described as being a composition which includes didecyl dimethyl ammonium chloride; BARDAC® LF and BARDAC® LF 80, which are described as being based on dioctyl dimethyl ammonium chloride; BARQUAT® MB-50, BARQUAT® MB-80, BARQUAT® MX-50, BARQUAT® MX-80, BARQUAT® OJ-50, BARQUAT® OJ-80, BARDAC® 208M, HYAMINE® 3500, HYAMINE® 3500-NF, BTC® 50, BTC® 824, BTC® 835, BTC® 885, BTC® 2565, BTC® 2658, BTC® 8248 or BTC® 8358 each described as being based on alkyl dimethyl benzyl ammonium chloride (benzalkonium chloride); BARQUAT® 4250, BARQUAT® 4280, BARQUAT® 4250Z, BARQUAT® 4280Z, BTC® 471, BTC® 2125, or BTC® 2125M, each described as being a composition based on alkyldimethylbenzyl ammonium chloride and/or alkyldimethylethylbenzyl ammonium chloride; BARQUAT® MS-100 or BTC® 324-P-100, each described as being based on myristyldimethylbenzyl ammonium chloride; HYAMINE® 2389, described as being based on methyldodecylbenzyl ammonium chloride and/or methyldodecylxylene-bis-trimethyl ammonium chloride; HYAMINE® 1622, described as being an aqueous solution of benzethonium chloride; as well as BARQUAT® 1552 or BTC® 776, described as being based on alkyl dimethyl benzyl ammonium chloride and/or dialkyl methyl benzyl ammonium chloride, BARQUAT® 50-MAB, described as being based on alkyldimethylethyl ammonium bromide and LONZABAC®-12.100, described as being based on an alkyl tertiary amine. (Each of these recited materials are presently commercially available from Lonza, Inc., Fairlawn, N.J. and/or from Stepan Co., Northfield, Ill.)

The biocidal constituent may be present in any effective amount, but generally need not be present in amounts in excess of about 10% wt. based on the total weight of the composition. The amount of biocide necessary to obtain an effective amount of sanitization is dependent on the surface area of the silica nano-particles. The biocidal cationic surfactant(s) may be present in the inventive food contact sanitizer compositions in amounts of from about 0.001% by weight to up to about 10% by weight, preferably about 0.05-5% by weight, more preferably in amount of between 0.01-2% by weight, and most preferably from 0.02-1% by weight It is particularly advantageous that the biocidal cationic surfactant(s) are present in amounts of at least 50 parts per million (ppm) to about 500 ppm.

Other Adjuncts:

Other optional adjuncts that may be employed in the inventive compositions herein include, but are not limited to: perfumes, fragrances and fragrance release agents, acids, electrolytes, dyes and/or colorants, solubilizing materials, wetting agents, solvents, stabilizers, thickeners, defoamers, hydrotropes, cloud point modifiers and preservatives.

Suitable solvents include mono-protic alkanols, multi-protic alcohols such as diols, alkyl and alkylene glycols, alkylene glycol ethers, selected carboxylic acids, and water soluble and water insoluble organic solvents. Alkanols include, but are not limited to, methanol, ethanol, n-propanol, isopropanol, butanol, pentanol, and hexanol, and isomers thereof. Diols include, but are not limited to, methylene, ethylene, propylene and butylene glycols. Alkylene glycol ethers include, but are not limited to, ethylene glycol monopropyl ether, ethylene glycol monobutyl ether, ethylene glycol monohexyl ether, diethylene glycol monopropyl ether, diethylene glycol monobutyl ether, diethylene glycol monohexyl ether, propylene glycol methyl ether, propylene glycol ethyl ether, propylene glycol n-propyl ether, propylene glycol monobutyl ether, propylene glycol t-butyl ether, di- or tri-polypropylene glycol methyl or ethyl or propyl or butyl ether, acetate and propionate esters of glycol ethers. Examples of organic solvents having a vapor pressure less than 0.1 mm Hg (20° C.) include, but are not limited to, dipropylene glycol n-propyl ether, dipropylene glycol t-butyl ether, dipropylene glycol n-butyl ether, tripropylene glycol methyl ether, tripropylene glycol n-butyl ether, diethylene glycol propyl ether, diethylene glycol butyl ether, dipropylene glycol methyl ether acetate, diethylene glycol ethyl ether acetate, and diethylene glycol butyl ether acetate (all available from ARCO Chemical Company). Short chain carboxylic acids include, but are not limited to, acetic acid, glycolic acid, lactic acid and propionic acid. Short chain esters include, but are not limited to, glycol acetate, and cyclic or linear volatile methylsiloxanes. Water insoluble solvents such as isoparafinic hydrocarbons, mineral spirits, alkylaromatics, terpenoids, terpenoid derivatives, terpenes, and terpenes derivatives can be mixed with a water soluble solvent when employed. Water-miscible organic solvents can be present in concentrations ranging from 0.1-5 wt. %.

Wetting agents include surfactants, surface-active agents, tensides, and silicone wetting and anti-foaming agents known in the art.

Acidic and basic pH modifiers may also be employed to adjust the pH of the inventive compositions. When used, common mineral acids and bases known in the art may be employed. Alternatively, organic acids and organic bases, and in particular organic base solvent buffers may be employed. Organic base solvent buffers include the alkanolamines, including monoethanolamine (MEA), diethanolamine (DEA) and triethanolamine (TEA), ammonia and ammonium derivatives. Organic base solvent buffers can be present in concentrations between 0.001-1 wt. %.

Experimental

The amount of quat per silica's surface area was determined according to particle size. Particles used included both smaller sizes (12 nm) with surface areas of 200 m²/g, and larger sizes (70 nm) with surface areas of 50 m²/g. Twelve samples were looked at:

TABLE 1

| SAMPLE | BIOCIDE | SILICA | RESULTS |
|---|---|---|---|
| Sample A | ¹Barquat 4250Z, 100 ppm | ²Bindzil 30/360FG | 0/10 |
| | | (12 nm), 0.075 ppm | |
| Sample B | ¹Barquat 4250Z, 100 ppm | ³Klebosol 35 V 50 | 0/10 |
| | | (70 nm), 0.10 ppm | |
| Sample C | ¹Barquat 4250Z, 100 ppm | None | 1/10 |
| Sample D | ¹Barquat 4250Z, 400 ppm | ²Bindzil 30/360FG | 0/10 |
| | | (12 nm), 0.075 ppm | |
| Sample E | ¹Barquat 4250Z, 400 ppm | ³Klebosol 35 V 50 | 0/10 |
| | | (70 nm), 0.10 ppm | |
| Sample F | ¹Barquat 4250Z, 400 ppm | None | 0/10 |

¹benzyl dialkyl quaternary ammonium chloride (Lonza)
²colloidal silica, particle size 12 nm (Akzo Nobel)
³colloidal silica, particle size 70 nm (Clariant)

Samples A-F were adjusted to pH 10.5 using a MEA buffer at 0.1 wt % and use-dilution tests ten carriers and log reduction were conducted with 10 minute contact times, E. Coli bacteria and no soil load for each sample.

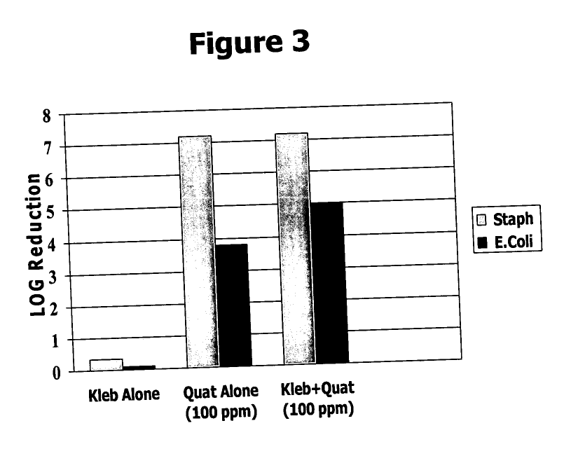

A food contact sanitization test was performed by ATS Laboratory Minneapolis, Minn. Colloidal silica particles of 70 nm at 100 ppm in water, the 4250Z quaternary ammonium at 100 ppm in water, and a combination of both at 100 ppm each in water were tested for micro efficacy of the Staph A. (56%), and E. Coli (27%) bacteria using the Suspension Methodology recommended by the EPA for food contact sanitization. To claim food contact sanitization both types of bacteria must be reduced by at least 5 logs as exhibited only the novel synergistic combination of the Pickering emulsion. Neutralization verification was also done in combination with a control at 700 ppm quaternary ammonium only. Results show that the silica alone didn't provide any log reduction of either bacterial count. Quaternary ammonium alone at 100 ppm was effective at reducing the gram-positive bacteria Staph by a factor of 10⁷ (7 logs), but ineffective at reducing gram-negative bacteria E. Coli by a factor of 1000 (3 logs). The combination of Barquat 4250Z quaternary ammonium (100 ppm) and 70 nm particles of suspended nano-colloidal silica (100 ppm) reduced effectively both the gram-positive bacteria by 7 logs, and the gram-negative bacteria by 5 logs. The results of the food contact sanitization test are summarized in FIG. 3.

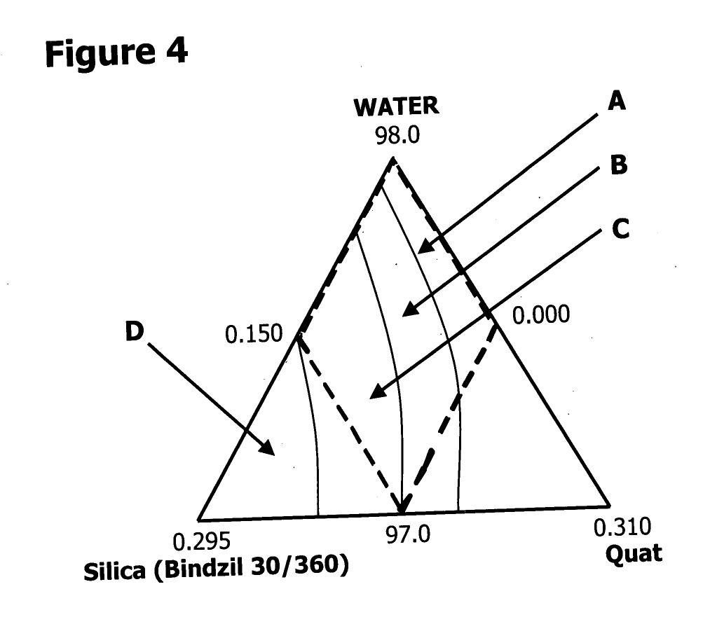

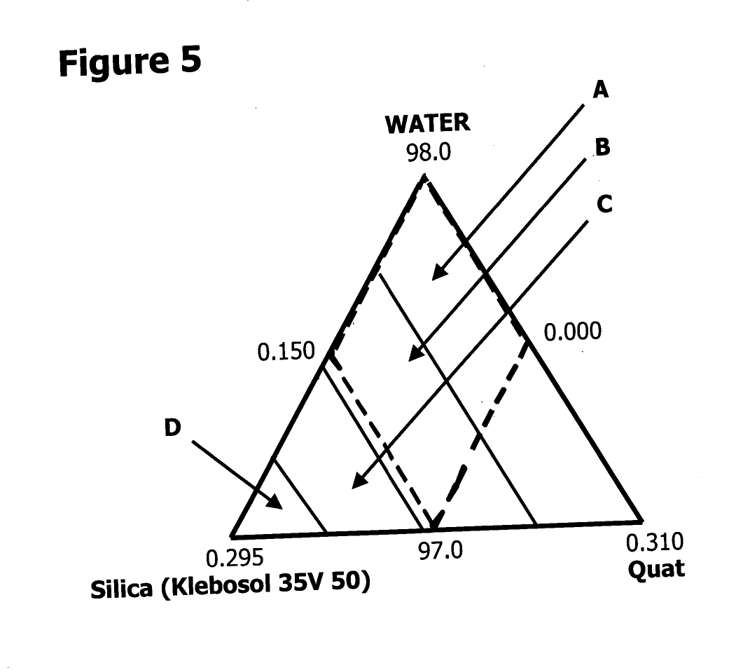

The amount of quat needed is generally dependent on the surface area of the silica gel particles chosen. The maximum (or preferred) amount of quat for a given type of silica is the combination that yields a one (single) phase solution (clear) of the Pickering emulsion, that is a solution which maintains the nano-particle sized silica in a fully dispersed suspension with little or no separation or precipitation of the nano-particles over the storage time of the composition. Generally, more concentrated compositions are preferred, which may be used directly on the targeted surface, or which may alternatively be diluted prior to use to form a use concentration appropriate to the particular application needed. Thus, compositions bordering on a two (dual) phase region (cloudy), that is concentrated to the greatest extent possible while still maintaining a single continuous phase are generally desirable. The phase boundary of a particular embodiment of the inventive nano-particle systems herein will generally depend on the nature of the materials employed, the ratio of the nanosilica component and the quaternary ammonium biocide as well as other optional adjuncts and additives which may influence solution properties, and may further be influenced by ambient conditions such as storage temperature. Thus, the phase boundary of any particular embodiment of the present invention is best determined experimentally to identify, with respect to the desired level of the quaternary biocidal active desired, the region encompassing the single phase region in which storage stable compositions may be selected. Referring to FIGS. 4 and 5, phase diagrams are shown which show mixture contour plots of Bindzil/Barquat 4250Z and Klebosol/Barquat 4250Z, respectively. The two phase diagrams resulted from 20 samples where the amount of silica was varied between 0-0.15% and the amount of Barquat 4250Z was varied between 0.015-0.16%. The dotted region of FIGS. 4 and 5 represents the formulation boundary corresponding to inventive compositions with the nano-sized silica present at between 0-0.15% and water from 97.7-98.0% in the example compositional embodiments of the present invention using two types of materials as described herein. Phase regions A, B, C and D enclose representative embodiment compositions that were observed to exhibit clear one-phase (A), cloudy one-phase (B), and cloudy two-phase (C) behavior, as well as formulations exhibiting visual precipitation (D).

Although the single phase compositions may be desirable for many applications, dual phase compositions may also be employed for particular applications, particularly if combined with means and/or instructions for an intermediate processing step, such as for example by additional dilution with water, to produce a suitable end-use formulation, which following said intermediate processing, is sufficiently modified in properties to be within the stable single phase region for a time sufficient for use in sanitizing the targeted surface. An illustrative example would be an embodiment in which a single phase composition is cooled upon storage, reverting to a separated system, but which may be reheated or diluted appropriately to provide a reconstituted single phase composition suitable for use at the time of application. In another embodiment, a single phase composition is applied to an absorbent substrate, such as a wipe intended for food contact sanitization use, and which separates to a dual phase composition owing to differential absorption by the absorbent carrier, and/or evaporation of the aqueous phase during processing, for example, but which upon wetting with water prior to use provides a wipe with the desired single phase composition suitable for the intended application.

This invention has been described herein to provide those skilled in the art with information relevant to apply the novel principles and to construct and use such specialized components as are required. However, it is to be understood that the invention can be carried out by different equipment, materials and devices, and that various modifications, both as to the equipment and operating procedures, can be accomplished without departing from the scope of the invention itself.

We claim:

1. A method of sanitizing a hard surface to come in contact with food comprising the steps of:
(1) contacting a hard surface with a spray containing or a non-woven wipe impregnated with a food contact sanitizer composition comprising: (a) an aqueous colloidal dispersion of nanoparticle-sized silica, wherein the average particle size of the silica particles ranges from 1 to 100 nm, and (b) at least one quaternary ammonium biocide, wherein the level of quaternary ammonium biocide present in said food contact sanitizer composition necessary to accomplish sanitization according to EPA standards is less than the level of quaternary ammonium biocide that would be necessary absent said aqueous colloidal dispersion of nanoparticle-sized silica; and
(2) wiping said sprayed surface or wiping said surface with the non-woven wipe such that a thin film residue of said food contact sanitizer composition remains on the hard surface, wherein in the absence of a rinsing step said quaternary ammonium biocide residue remaining on the surface is less than 200 ppm.

2. A hard surface food contact sanitizer composition comprising:
an aqueous colloidal dispersion of nanoparticle-sized silica, wherein the average particle size of the silica particles ranges from 1 to 100 nm; and
at least one quaternary ammonium biocide, wherein the level of quaternary ammonium biocide present in the food contact sanitizer composition necessary to accomplish sanitization according to EPA standards is less than the level of quaternary ammonium biocide that would be necessary absent the colloidal dispersion of nanoparticle-sized silica.

3. The hard surface food contact sanitizer composition recited in claim 2, wherein the ratio of silica to quaternary ammonium ranges from 5×10⁻⁵:1 to 1.:1.

4. The hard surface food contact sanitizer composition recited in claim 2, wherein the silica is amorphous silica.

5. The hard surface food contact sanitizer composition recited in claim 2, wherein the silica has a particle size distribution ranging from 5 to 150 nm.

6. The hard surface food contact sanitizer composition recited in claim 2, wherein the silica has a surface area ranging from 50 to 800 m²/g.

7. The hard surface food contact sanitizer composition recited in claim 2, wherein the quaternary ammonium biocide is selected from the group consisting of quaternary ammonium compounds and salts thereof, which may be characterized by the general structural formula:
(R1R2R3R4)N+X−
wherein each of R1, R2, R3 and R4 is independently selected from an alkyl, aryl, alkylaryl or alkoxylated substituent of from 1 to 26 carbon atoms, and the entire cation portion of the molecule has a molecular weight of at least 165, wherein X− is any suitable anion.

8. The hard surface food contact sanitizer composition recited in claim 7, wherein the quaternary ammonium compound is a dialkyl ammonium quaternary biocide.

9. The hard surface food contact sanitizer composition recited in claim 2, wherein the concentration of silica ranges from 0.05 to 10 ppm.

10. The hard surface food contact sanitizer composition recited in claim 2, wherein the concentration of biocide ranges from 10 to 1000 ppm.

11. The hard surface food contact sanitizer composition recited in claim 2, further comprising a wetting agent.

12. The hard surface food contact sanitizer composition recited in claim 11, wherein the wetting agent is selected from the group consisting of surfactants, surface-active agents, tensides, silicone wetting agents, silicone anti-foaming agents, and mixtures thereof.

13. The hard surface food contact sanitizer composition recited in claim 11, wherein the wetting agent is a silicone based wetting agent.

14. The hard surface food contact sanitizer composition recited in claim 2, further comprising a water-miscible organic solvent.

15. The hard surface food contact sanitizer composition recited in claim 14, wherein the amount of water-miscible organic solvent ranges from 0.1 to 5 wt %.

16. The hard surface food contact sanitizer composition recited in claim 15, wherein the water-miscible organic solvent is selected from the group consisting of alcohols, diols, glycols, glycol ethers, alkylene glycol ethers, and mixtures thereof.

17. The hard surface food contact sanitizer composition recited in claim 16, wherein the alcohol is ethanol or isopropanol.

18. The hard surface food contact sanitizer composition recited in claim 2, further comprising an organic base solvent buffer selected from the group consisting of monoethanolamine, diethanolamine, triethanolamine, ammonia, ammonium derivatives, and mixtures thereof, wherein the amount of organic base solvent buffer ranges from 0.001 to 1.0 wt %.

19. The hard surface food contact sanitizer composition recited in claim 2, further comprising a non-woven substrate.

20. A hard surface food contact sanitizer composition comprising:
an aqueous colloidal dispersion of nanoparticle-sized silica, wherein the silica particles have a particle size distribution range of 1-100 nm; and
at least one quaternary ammonium biocide such that the concentration of silica ranges from 0.05 to 10 ppm and the concentration of biocide ranges from 10 to 1000 ppm.
